(19) United States
(12) Patent Application Publication
(10) Pub. No.: US 2022/0313659 A1
(43) Pub. Date: Oct. 6, 2022

(54) Methods for Increasing the Bioavailability of OTC and Pharmaceutical Drugs

(71) Applicant: LOCUS IP COMPANY, LLC, Solon, OH (US)

(72) Inventors: Sean FARMER, Ft. Lauderdale, FL (US); Ken ALIBEK, Solon, OH (US)

(21) Appl. No.: 17/634,172

(22) PCT Filed: Aug. 10, 2020
(86) PCT No.: PCT/US2020/045587

§ 371 Date: Feb. 9, 2022

Related U.S. Application Data
(60) Provisional application No. 62/885,233, filed on Aug. 10, 2019.

(51) Int. Cl. A61K 31/4045; A61K 31/7056; A61K 45/06; A61K 47/42; A61K 47/36

(52) U.S. Cl. CPC A61K 31/4045; A61K 31/7056; A61K 45/06; A61K 47/42; A61K 47/36

(57) ABSTRACT

The present invention relates to compositions and methods for enhancing the efficacy of drug compounds. The subject invention utilizes an adjuvant composition comprising of one or more microbial-produced biosurfactants and/or isoforms thereof to enhance bioavailability, stability, and localization of the drugs in a subject, to reduce the total dosage that is required, and to prolong the pre-administration stability of the drugs.

### CROSS-REFERENCE TO RELATED APPLICATION

This application claims priority to U.S. Provisional Patent Application No. 62/885,233, filed Aug. 10, 2019, which is incorporated herein by reference in its entirety.

### BACKGROUND OF THE INVENTION

Bioavailability is critically important to the efficacy of a pharmaceutical or over-the-counter (OTC) drug compound. Bioavailability is defined as the amount of an administered compound that reaches circulation, unchanged, in a subject. Compounds that are injected intravenously are defined as entirely bioavailable; however, there are a few primary factors that affect bioavailability of other methods of administration: solubility, stability, and membrane or tissue permeability. Additionally, some drugs fail to localize in the desired site because of an inability to move from systemic circulation through the blood-brain barrier or the blood-testis barrier. Each factor, independently or dependently, affects the amount of unchanged drug delivered to the target cell, tissue, organ, or fluid.

Some drugs have poor solubility in water. A compound is generally considered to be poorly soluble if it is soluble in a concentration less than 10 mg/ml in an aqueous solution at a neutral pH at room temperature. Examples of poorly soluble drugs include ibuprofen, naproxen, and indinavir. The increased risk for kidney stones due to crystallized, insoluble indinavir is so great that it is not currently recommended for use to treat HIV.

Epithelial tissues are another barrier to numerous drugs. The inability to permeate gastrointestinal or other epithelial tissues can be caused by factors other than solubility, which include the active secretion of the drug or failure to traverse the junctions between epithelial cells.

The blood-brain barrier (BBB) has tight junctions between cells that inhibit most drug compounds from passing through the tissue. When drugs are able to move through the BBB or epithelial tissues, transport mechanisms can secrete them back out of the BBB or systemic circulation, respectively.

The efficacy of some drug compounds can benefit from a sustained release in a subject because of short elimination half-lives. Metformin is used to treat type 2 diabetes, and capecitabine (CB) is a synthetic drug that is used to treat various cancers including breast, gastric, prostate, and colorectal. The efficacy of CB and metformin is affected by short elimination half-lives. Increasing the elimination half-life increases a subject's exposure time to the drugs. Many orally delivered antibiotics have to be taken multiple times per day for weeks, leading to non-adherence to the prescribed antibiotic course. A more sustained release of a drug can decrease the number of doses taken, increasing the likelihood of patient adherence to the prescribed drug treatment course. Other drugs are not stable upon oral administration or can present toxic symptoms as observed upon the oral delivery of testosterone.

Chemical surfactants are regularly used in pharmaceutical and OTC compositions. Existing chemical surfactants have drawbacks including toxicity and wastewater pollution during disposal. Sodium dodecyl sulfate (SDS) is a chemical surfactant that is extensively used in drug compositions to increase the permeability of the blood-brain barrier, increase the solubility of ibuprofen, and increase the flux of azithromycin through mouse skin. However, SDS, like other chemical surfactants, can irritate skin by altering the protein kinase C cell signal transduction cascade. Additionally, wastewater discharged from hospitals often contains glutaraldehyde, which is used as a disinfectant in conjunction with surfactants such as SDS and cetyl trimethyl ammonium bromide (CTAB). SDS is toxic to invertebrates and crustaceans; toxicity increases when combined with glutaraldehyde.

Thus, there is a need for safe, effective compositions and methods of improving the efficacy of a wide variety of OTC and pharmaceutical compounds.

### BRIEF SUMMARY OF INVENTION

The subject invention provides compositions and methods for improving the efficacy of pharmaceutical and OTC compounds using microbial-sourced biosurfactants. Additionally, these biosurfactants facilitate the cost-effective preparation of drug compositions that are non-toxic.

In one embodiment, the present invention provides therapeutic compositions comprising an active component and an adjuvant component, wherein the active ingredient can be a pharmaceutical and/or OTC drug, for example, daptomycin, clindamycin, azithromycin, moxifloxacin, bortezomib, lenalidomide, abiraterone acetate, pegfilgrastim, capecitabine, doxorubicin, erlotinib, aspirin, naproxen, ibuprofen, metformin, donepezil, nitazoxanide, varenicline, testosterone, sildenafil, vardenafil, tadalafil, or indinavir, or any alternative form of the aforementioned compounds.

In certain embodiments, the active component is a vitamin, mineral, supplement, herbal extract, or other health-promoting substance.

In preferred embodiments, the bioavailability, stability, and/or localization of the active component are enhanced through the use of an adjuvant component of the subject invention comprising one or more microbial-sourced biosurfactants.

In certain embodiments, methods are provided for improving the bioavailability, stability and/or localization of an active component, i.e., a drug, wherein the active component is administered to a subject simultaneously with the adjuvant component or within, for example, 5 minutes before or after administering the adjuvant component.

The use of biosurfactants with drug compounds can also have effects other than enhancing bioavailability, stability, and localization of the drugs in a subject and decreasing the amount of chemical surfactants used in drug compositions. For example, in some embodiments, biosurfactants can increase the shelf life and stability of a drug composition before administration to a subject, particularly for drugs that are poorly soluble in aqueous solutions. The biosurfactants enable the drug compounds to remain dissolved in an aqueous solution and resist elevated temperature and UV light exposure.

In certain embodiments, the biosurfactants of the subject compositions can be a glycolipid biosurfactant or a lipopeptide biosurfactant. In specific preferred embodiments, the glycolipid is selected from sophorolipids, rhamnolipids, trehalose lipids, cellobiose lipids, and mannosylerythritol lipids; and the lipopeptide is selected from surfactins, iturins, lichenysins, and fengycins. In certain embodiments, the sophorolipid is an acidic sophorolipid, with a structure comprising a sophorose carbohydrate head and fatty acid tail that is 16 or 18 carbon atoms in length.

In certain embodiments, the lipopeptide is surfactin, with a structure comprising a peptide loop of seven amino acids and a hydrophobic fatty acid chain that is thirteen to fifteen carbons long. In specific embodiments, the amino acids comprise L-aspartic acid, L-glutamic acid, two L-leucine, two D-leucine, and L-valine.

In certain embodiments, the lipopeptide is an inturin. Iturins have a variety of amino acid residues that make up the peptide moiety and a variety of fatty acids or fatty acid derivatives that make up the hydrophobic tail of the biosurfactant. In preferred embodiments, the iturin is iturin A, comprising a peptide loop of seven amino acids, which are two D-asparagines, L-asparagine, D-tyrosine, L-glutamine, L-proline, and L-serine, and a t3-amino fatty acid chain that can be fourteen to seventeen carbons long.

In certain embodiments, the biosurfactants are present in the subject composition in critical micelle concentration (CMC). In certain embodiments, the composition further comprises one or more pharmaceutical carriers. In other embodiments, the composition further comprises a biosurfactant-based nanoparticle delivery system.

In preferred embodiments, the subject therapeutic compositions are formulated and administered as orally-consumable products, such as, for example food items, capsules, pills, and drinkable liquids. The compositions of the subject invention can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be formed and administered sublingually, buccally, rectally, or vaginally. Furthermore, the compositions can be formed for nebulization, spraying into the nose for absorption through the nasal membrane, inhalation via the mouth or nose, or administration in the eye or ear.

In certain embodiments, the biosurfactants of the subject compositions increase the solubility of drugs both in the subject and in aqueous solutions for administration. For example, the solubility of nitazoxanide, erlotinib, abiraterone acetate, bortezomib, azithromycin, moxifloxacin, indinavir, ibuprofen, naproxen, aspirin, testosterone, vardenafil, tadalafil, sildenafil, lenalidomide, or any alternative for these pharmaceuticals can be increased. Each of these drugs has a maximum solubility in water at room temperature of less than 10 mg/ml.

In another embodiment, the biosurfactants of the subject compositions decrease the total dose of surfactant molecules and/or drug compounds delivered to the subject. Even for those drugs that have solubilities greater than 10 mg/ml, the addition of a biosurfactant can decrease the amount of chemical surfactants, such as poloxamers and SDS, used in compositions with pharmaceuticals and OTC medications.

In another embodiment, the biosurfactants of the subject compositions increase the permeability of drugs through epithelial tissues. For example, the permeability of lenalidomide, abiraterone acetate, capecitabine, metformin, nitazoxanide and/or other drugs can be increased. Each of these drugs is frequently prescribed, yet each drug inefficiently crosses the intestinal epithelial cell barriers to move into systemic circulation.

In another embodiment, the biosurfactants of the subject compositions increase the elimination half-life of drugs, thereby decreasing the frequency of dose administration. For example, the elimination half-life of capecitabine, clindamycin, metformin, nitazoxanide, indinavir and/or other drugs can be increased. Each of these drugs has a short elimination half-life, and is usually prescribed to be taken multiple times per day (e.g., as many as 4 times per day) to maintain therapeutic levels of the drugs in the subject.

In another embodiment, the biosurfactants of the subject compositions prolong the release time of a drug in the subject. This prolonging of drug release is facilitated by drugs entrapped in micelles or other biosurfactant-based drug delivery systems. For example, the release time of daptomycin, clindamycin, azithromycin, moxifloxacin, lenalidomide, abiraterone acetate, capecitabine, erlotinib, aspirin, naproxen, ibuprofen, metformin, donepezil, nitazoxanide, varenicline, sildenafil, tadalafil, indinavir and/or other drugs can be prolonged. Each of these drugs is, or can be, taken at least once daily. By using biosurfactant micelles or biosurfactant-based drug delivery, including nanoparticles, liposomes, or nanoemulsion droplets, the drug can be released for a prolonged period as the relative concentration of biosurfactant decreases after the composition is administered to a subject. This works to decrease the number of doses a subject takes to achieve a therapeutic effect.

In another embodiment, the biosurfactants of the subject compositions improve the movement of a drug through the blood-brain barrier (BBB). For example, BBB transversal of bortezomib, lenalidomide, donepezil, varenicline, nitazoxanide, indinavir, sildenafil, vardenafil, tadalafil and/or other drugs can be improved. Each of these drugs has a therapeutic effect within the BBB, but the BBB is a prominent barrier to entry to the cerebrospinal fluid (CSF) from systemic circulation. The blood-testis barrier is also a significant impediment to drugs such as indinavir. With biosurfactants, indinavir can access the blood-testis barrier more readily and interact with retroviruses present in the testes. However, if a drug can move into the CSF, P-glycoprotein (P-gp) readily pumps out drugs that are substrates of this ATP-binding cassette (ABC) transport protein.

In another embodiment, the biosurfactants of the subject compositions inhibit secretion systems, increasing the time that a drug remains within the CSF. The presence of biosurfactant changes the membranes in which these secretion systems reside, altering the efficacy of the pumps. For example, the time that indinavir, clindamycin, daptomycin, moxifloxacin, bortezomib, lenalidomide, capecitabine, doxorubicin, erlotinib, sildenafil, vardenafil, tadalafil and/or other drugs remains in the CSF can be increased. P-gp is a common example of the secretion system that can be inhibited from secreting the aforementioned drugs, but there are other examples of drug-secreting efflux pumps.

In another embodiment, the biosurfactants of the subject compositions decrease the time to achieve the maximum drug concentration in a subject. For example, donepezil is used to treat Alzheimer's Disease and must enter the CSF to be effective. However, in the treatment protocols known in the current art, donepezil does not achieve a steady state concentration for 3 months and a 50% increase in the concentration in a subject is observed between the 12 and 24 months after treatment commencement.

In certain embodiments, the biosurfactants of the subject composition reduce or eliminate the use of chemical surfactants. In some embodiments, the biosurfactants of the subject composition decrease the modifications of a drug in a subject after administration; modifications can be caused by, for example, acids in the GI tract or bound proteins.

In another embodiment, the biosurfactants of the subject composition increase the stability of a drug before administration to a subject, potentially removing the strict requirements for cold chain. This provides a greater ability to distribute drugs to, for example, regions that lack sufficient infrastructure for traditional cold chain storage.

Advantageously, the materials and methods of the subject invention can improve the efficacy of numerous pharmaceutical and OTC drugs for subjects in need thereof without the consequences of using chemical surfactants.

### DETAILED DISCLOSURE OF INVENTION

The subject invention provides materials and methods for enhancing the bioavailability, stability and/or localization of pharmaceutical and/or OTC drugs. In particular, the subject invention provides therapeutic compositions with microbial-sourced biosurfactants for use in enhancing the efficacy of active components, such as drug compounds. The resulting compositions and methods of the invention are non-toxic and cost-effective, and, advantageously, can help decrease the use of chemical surfactants in pharmaceuticals and OTC drugs.

Further described herein are methods for enhancing bioavailability of a drug through various means, which include, for example, increasing the solubility, elimination half-life, and/or permeability of a drug through epithelial tissues; prolonging the delivery time period of a drug; and/or improving stability of a drug prior to administration to a subject. In specific exemplary embodiments, the compositions of the subject invention, when administered to a subject, improve the localization of drugs that operate by suppressing P-glycoprotein (P-gp) secretion and other secretion systems, allowing a greater penetration of the blood-brain barrier and blood-testis barrier.

### Selected Definitions

As used herein, the term “adjuvant” means an auxiliary compound that can aid in, contribute to, and/or enhance the effectiveness of a substance that is administered with the adjuvant. For example, an adjuvant can be taken alongside a drug compound, and/or included in a therapeutic composition comprising a drug compound, to aid in the effectiveness of the drug for whatever its purpose may be (e.g., treating symptoms of a disease, or enhancing the functioning of an organ or system in the body).

As used herein, a “biofilm” is a complex aggregate of microorganisms, such as bacteria, wherein the cells adhere to each other using a matrix usually composed of, but not limited to, polysaccharide material. The cells in biofilms are physiologically distinct from planktonic cells of the same organism, which are single cells that can float or swim in liquid or gaseous mediums, or reside on or in solid or semi-solid surfaces. Individual microbial cells can also be filamentous, banding together in chains of cells, without forming distinct biofilms. Although, the filamentous attributes of the cells can facilitate the creation of biofilms.

As used herein, a “drug” refers to a compound manufactured, produced, extracted or otherwise obtained for use as a medicinal and/or therapeutic agent. Drugs can be any molecule or molecules that are meant to be delivered into blood and/or lymphatic circulation, tissues, or organs, ultimately reaching a site in a subject's body where a positive impact on the subject's health, either locally or systemically, can be effected. Drugs can be “pharmaceutical” drugs, meaning requiring a prescription from a health care provider in order to obtain, or “over-the-counter (OTC),” meaning available for purchase without a prescription. In certain embodiments, “drugs” can also include health-promoting substances, such as vitamins, minerals, supplements sources of amino acids (including essential amino acids and branched-chain amino acids), peptides, proteins, microelements, fats, fatty acids, lipids, carbohydrates, sterols, polyketides, biopolymers, herbal extracts and enzymes.

Drugs can include, for example, agents used for relieving pain, fever, and/or inflammation, reducing the symptoms of allergies or colds, suppressing or treating a virus, treating a bacterial or eukaryotic infection, treating cancer or the alleviating the effects or traditional cancer treatments, suppressing or preventing seizures, lowering or managing cholesterol, managing diabetes, treating depression or anxiety, controlling body weight, reducing or enhancing fertility, treating or alleviating the effects dementia, which includes Alzheimer's disease, treating or alleviating the effects erectile dysfunction, treating alleviate the effects of pulmonary arterial hypertension, treating addiction to chemicals, or treating or alleviating the effects hypogonadism.

As used herein, reference to a “microbe-based composition” or “microbial-sourced composition” means a composition that comprises components that were produced as the result of the growth of microorganisms or other cell cultures. A microbe-based composition may comprise the microbes themselves, or the microbes may be separated from the broth or media in which they were cultivated. The composition may comprise residual cellular components and/or by-products of microbial growth. The by-products of microbial growth may be, for example, metabolites (e.g., biosurfactants), cell membrane components, synthesized proteins, and/or other cellular components. Preferably, the therapeutic compositions according to the subject invention do not comprise microbe-based compositions that contain any microbes.

The subject invention further provides “microbe-based products,” which are products that are to be applied in practice to achieve a desired result. The microbe-based product can be simply a microbe-based composition harvested from a microbe cultivation process. Alternatively, the microbe-based product may comprise further ingredients that have been added. These additional ingredients can include, for example, stabilizers, buffers, and/or appropriate carriers (e.g., water or salt solutions). The microbe-based product may comprise mixtures of microbe-based compositions. The microbe-based product may also comprise one or more components of a microbe-based composition that have been processed in some way such as, but not limited to, filtering, centrifugation, lysing, drying, purification, and the like.

As used herein, an “isolated” or “purified” compound is substantially free of other compounds, such as cellular material, with which it is associated in nature or in which it was produced. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is, preferably, at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate, or an end product of metabolism. Examples of metabolites include, but are not limited to, biosurfactants, enzymes, acids, solvents, gases, alcohols, proteins, vitamins, minerals, microelements, amino acids, and polymers.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity or extent of the onset of such a condition, disease, or disorder, and/or inhibiting the progression of the condition, disease, or disorder to a more severe or extensive condition, disease, or disorder.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 1%, 5%, 10%, 25%, 50%, 75%, or 100%. By “increases” is meant as a positive alteration of at least 1%, 5%, 10%, 25%, 50%, 75%, or 100%.

By “reference” is meant a standard or control condition.

As used herein, the term “subject” refers to an animal needing or desiring delivery of the benefits provided by a drug compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. The benefits can include, but are not limited to, the treatment of a health condition, disease or disorder; prevention of a health condition, disease or disorder; enhancement of immune health; and/or enhancement of the function of an organ, tissue, or system in the body. The preferred subject in the context of this invention is a human. In some embodiments, a subject is suffering from a health condition, disease, or disorder, while in some embodiments, the subject is in a state of good health (e.g., substantially free from injury or illness) but desires enhanced health and/or functioning of a particular organ, tissue, or body system. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or preventing a condition, disease, or disorder, or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or prevented; the severity of the condition; the particular organ, tissue, or body system of which enhancement in health or function is desired; the weight, height, age, and health of the patient; and the route of administration.

As used herein, “surfactant” refers to a surface-active substance that lowers the surface tension (or interfacial tension) between phases. Surfactants act as, for example, detergents, wetting agents, emulsifiers, foaming agents, and/or dispersants. A surface-active substance produced by microorganisms is referred to as a “biosurfactant.”

As used herein, the term “treatment” refers to eradicating, reducing, ameliorating, improving or reversing a sign or symptom of a health condition, disease or disorder to any extent, and includes, but does not require, a complete cure of the condition, disease, or disorder. Treatment can be curing or partially ameliorating a condition, disease or disorder. “Treatment” can also include improving or enhancing a condition or characteristic, for example, bringing the function of a particular system in the body to a heightened state of health or homeostasis.

The transitional term “comprising,” which is synonymous with “including,” or “containing,” is inclusive or open-ended and does not exclude additional elements or method steps not recited. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates other embodiments that “consist” and/or “consist essentially” of the recited element(s).

Unless specifically stated or is obvious from context, as used herein, the term “or” is understood to be inclusive. Unless specifically stated or is obvious from context, as used herein, the terms “a,” “an” and “the” are understood to be singular or plural.

Unless specifically stated or is obvious from context, as used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Formulation and Delivery of Therapeutic Compositions

The subject invention provides therapeutic compositions comprising microbial-sourced biosurfactants for use in enhancing the efficacy of drugs. Advantageously, the compositions and methods of the subject invention are non-toxic and cost-effective.

More specifically, in certain embodiments, the therapeutic compositions comprise an active component and an adjuvant component, the active component comprising one or more pharmaceutical or OTC drugs; wherein said adjuvant component comprises an efficacy-enhancing amount of a biosurfactant; and wherein bioavailability, localization, and/or stability of the drug is enhanced compared to a composition comprising the same drug without the adjuvant component.

The active component according to the subject invention can be any drug, including pharmaceutical and OTC drugs. For example, the active component can be moxifloxacin, bortezomib, lenalidomide, abiraterone acetate, pegfilgrastim, capecitabine, doxorubicin, erlotinib, aspirin, naproxen, ibuprofen, donepezil, nitazoxanide, varenicline, testosterone, sildenafil, vardenafil, tadalafil, indinavir, ribavirin, metformin, and any alternative forms thereof,

such as, for example, clindamycin phosphate, clindamycin hydrochloride, clindamycin palmitate hydrochloride, azithromycin dihydrate, moxifloxacin hydrochloride, abiraterone, filgrastim, doxorubicin hydrochloride, PEGylated doxorubicin in liposome, non-PEGylated doxorubicin in liposome, PEGylated doxorubicin, erlotinib hydrochloride, naproxen sodium, ibuprofen sodium, donepezil hydrochloride, varenicline tartrate, testosterone undecanoate, testosterone cypionate, testosterone enanthate, testosterone propionate, sildenafil citrate, vardenafil HCl, vardenafil HCl trihydrate, and indinavir sulfate.

Other active components can include, for example, acetaminophen, benzoyl peroxide, neomycin, polymyxin, calamine (zinc oxide/ferric oxide), salicylic acid, dimethicone, hydrocortisone (cortisol), sunscreen (e.g., oxybenzone, avobenzone, octisalate, octocrylene, homosalate, or octinoxate), malathion, permethrin, antacids/proton-pump inhibitors (e.g., bismuth subsalicylate, famotidine, lansoprazole, ranitidine hydrochloride, omepraole, calcium carbonate), loperamide, glucose, insulin, meclizine, antihistamines (e.g., brompheniramine, cetirizine, chlorpheniramine, clemastine, diphenhydramine, fexofenadine, loratadine), guaifenesin, destromethorphan, oxymetazoline, phenylephrine, pseudoephedrine, lotrimin, miconazole, clotrimazole, tinactin, ketoconazole, benzocaine, and menthol.

Further additional active components can include various antibiotics, including, for example, penicillins (such as penicillin G, penicillin V, ampicillin, amoxicillin, bacampicillin, carbenicillin, carbenicillin indanyl, ticarcillin, azlocillin, mezlocillin, methicillin, piperacillin, and the like), tetracyclines (such as chlortetracycline, oxytetracycline, methacycline, doxycycline, minocycline and the like), cephalosporins (such as cefadroxil, cephalexin, cephradine, cephalothin, cephapirin, cefazolin, cefaclor, cefamandole, cefonicid, cefoxitin, cefotetan, cefuroxime, cefuroxime axetil, cefinetazole, cefprozil, loracarbef, ceforanide, cefepime, cefoperazone, cefotaxime, ceftizoxime, ceftriaxone, ceftazidime, cefixime, cefpodoxime, ceftibuten, and the like), fluoroquinolones (e.g., levofloxacin), quinolones (such as nalidixic acid, cinoxacin, ciprofloxacin and norfloxacin and the like), lincomycins (e.g., clindamycin), macrolides (e.g., erythromycin, azithromycin), sulfones (e.g., dapsone), sulfonamides (e.g., sulfanilamide, sulfadiazine, sulfamethoxazole, sulfisoxazole, sulfacetamide, bactrim), lipopeptides (e.g., daptomycin), polypeptides (e.g., bacitracin), glycopeptides (e.g., vancomycin), aminoglycosides (e.g., streptomycin, gentamicin, tobramycin, amikacin, netilmicin, kanamycin, and the like), nitoimidazoles (e.g., metronidazole) and/or carbapenems (e.g., thienamycin).

Yet further additional examples can include muscle relaxants; digestive aids (e.g., reflux suppressants, laxatives, probiotics, prebiotics, and antidiarrheals); cardiovascular drugs (e.g., beta blockers, calcium channel blockers, diuretics, vasoconstrictors, vasodilators, cardiac glycosides, antiarrhythmics, nitrates); blood pressure/hypertension drugs (e.g., ACE inhibitors, alpha blockers, angiotensin receptor blockers); coagulation drugs (e.g., anticoagulants, heparin, antiplatelet drugs, fibrinolytics, anti-hemophilic factors and haemostatic drugs); statins (e.g., LDL cholesterol inhibitors and hypolipidaemic agents); endocrine aids (e.g., androgens, antiandrogens, estrogens, gonadotropin, corticosteroids, HGH, vasopressin); antidiabetics (e.g., sulfonylureas, biguanides, metformin, thiazolidinediones, insulin); thyroid hormones and antithyroid drugs; urogenital system drugs (e.g., antifungals, alkalinizing agents, quinolones, antibiotics, cholinergics, anticholinergics, fertility medications, hormonal contraceptives); central nervous system drugs (e.g., psychedelics, hypnotics, anesthetics, antipsychotics, eugeroics, antidepressants (including tricyclics, monoamine oxidase inhibitors, lithium salts, and SSRIs), antiemetics, anticonvulsants/antiepileptics, stimulants, amphetamines, dopamine agonists, antihistamines, cannabinoids, 5-HT antagonists); ocular medications (e.g., topical anesthetics, sympathomimetics, parasympatholytics, mydriatics, cycloplegics, mast cell inhibitors); antimicrobials (e.g., antibiotics, antibacterials, antifungals, antiparasitics, antiprotozoals, amoebicides); antivirals (e.g., acyclovir, ribavirin, valacyclovir, famciclovir, ganciclovir), antihistamines, anticholinergics, antiseptics, cerumenolytics, bronchodilators, antitussives, mucolytics, decongestants, antimalarials, antitoxins, antivenoms, vaccines, immunoglobulins, immunosuppressants, interferons, monoclonal antibodies, chemotherapeutic drugs and/or any other category of compounds that are capable of treating any health condition, disease or disorder, or of enhancing health in any way.

In some embodiments, the therapeutic composition comprises one or more other health-promoting substances, such as vitamins, minerals, and/or supplements. These other substances can include, for example, sources of amino acids (including essential amino acids and branched-chain amino acids), peptides, proteins, microelements, fats, fatty acids, lipids, carbohydrates, sterols, polyketides, biopolymers, herbal extracts and enzymes.

In certain embodiments, the other health-promoting substance is the active component in the therapeutic composition. In other embodiments, the other health-promoting substance is present in addition to a pharmaceutical or OTC active component, such as those listed above.

In one embodiment, the health-promoting substance is a vitamin, such as, for example, vitamins A, C, D, E, K, B1 (thiamine), B2 (riboflavin), B3 (niacin), B6, B7 (biotin), B12, folate (or folic acid), panthothenic acid, nicotinic acid, choline chloride, carnitine, inositol and para-amino-benzoic acid. In certain embodiments, the adjuvant composition can help facilitate solubilization of lipophilic vitamins, such as, for example, vitamins A, D, E, and/or K.

In one embodiment, the health-promoting substance is a macro-minerals and/or trace mineral, such as, for example, calcium, phosphorus, magnesium, sodium, potassium, chloride, sulfur, iron, manganese, copper, iodine, zinc, cobalt, fluoride and selenium.

In one embodiment, the health-promoting substance is a supplement, such as, for example, caffeine, Echinacea, fish oil, Ginseng, glucosamine, chondroitin sulfate, garlic extract, St. John's Wort, Saw Palmetto, ginko, omega-3 fatty acids, omega-6 fatty acids, melatonin, beta carotene, flavonoids (e.g., anthocyanins), collagen peptides, acai, activated charcoal, alfalfa, Arnica, Astragalus, aloe vera, ashwagandha, bee pollen, Belladonna, berberine, bilberry, betaine, bitter melon, bitter orange, black cohosh, black Psyllium, black tea, blessed thistle, blond Psyllium, blueberry, blue-green algae, boron, butterbur, calendula, cannabidiol (CBD), capsaicin, Capsicum, cartilage, cat's claw, chamomile, chasteberry, chitosan, cinnamon, clove, coconut, cod liver oil, colloidal silver, cranberry, creatine, dandelion, deer velvet, devil's claw, DHEA, Dong Quai, eleuthero, ephedra, Eucalyptus, elderberry, evening primrose, fenugreek, feverfew, flaxseed, Fucus vesiculosus, ginger, glycyrrhizin, goji, goldenseal, grape, grape seed, grapefruit, green coffee, green tea, guarana, guar gum, gymnema, hawthorn, hemp, hibiscus, honey, honokiol, hoodia, hops, horse chestnut, horny goat weed, horsetail, hydrazine sulfate, kava, kola nut, lavender, lemongrass, licorice root, lutein, lycopene, maca, mangosteen, methylsulfonylmethane, milk thistle, mistletoe, monolaurin, niacinamide, noni, oats, olive, oregano, palm oil, Papaya, pau d′arco, peanut oil, pennyroyal, peppermint, pomegranate, propolis, quercetin, rose hip, raspberry ketone, red clover, red yeast rice, reishi mushroom, resveratrol, rose hip, sage, saw palmetto, Satureja bachtiarica oil, Senna, slippery elm, soy, spearmint, Stevia, tart cherry, tea tree oil, thunder god vine, beetroot, tellimagrandin II, turmeric, valerian, whey protein, wild yam, willow bark, yerba mate, yohimbe, 5-HTP and others.

In one embodiment, the health-promoting substance is an enzyme, such as, for example, nattokinase, coenzyme Q10, lipase, bromelain, papain, chymopapain A, chymopapain B, Papaya peptidase A, trypsin, chymotrypsin, proteases, lipases, amylases, pancrelipase, digestive enzymes, lactase, alpha-glactosidase, cellulase, phytase, and beta-glucanase.

Other health-promoting substances may include, but are not limited to, antioxidants, beta-glucans, bile salt, cholesterols, carotenoids, and many others.

In certain embodiments, the subject compositions comprise an adjuvant compositions for enhancing the bioavailability, stability, and/or localization of a drug, wherein the adjuvant compositions comprise one or more biosurfactants in efficacy-enhancing amounts. In preferred embodiments, an “efficacy-enhancing amount” is an amount of the adjuvant composition (or adjuvant component of the subject therapeutic compositions) that improves the performance and/or effectiveness of the drug (or active component of the subject therapeutic compositions), when compared with other compositions comprising the same drug without the subject adjuvant component.

The adjuvant component comprises biosurfactants, which are a structurally diverse group of surface-active substances produced by microorganisms. Biosurfactants are safe, biodegradable and can be produced with ease at low cost using selected organisms in or on renewable substrates.

All biosurfactants are amphiphilic. They consist of two parts: a polar (hydrophilic) moiety and a non-polar (hydrophobic) group. Due to their amphiphilic structure, biosurfactants can increase the surface area of hydrophobic, water-insoluble substances, increase the water solubility of such substances, and change the properties of bacterial cell membranes. Biosurfactants accumulate at interfaces and reduce the surface and interfacial tension between molecules of liquids, solids, and gases, leading to the formation of aggregated micellular structures in solution once the concentration reaches a critical micelle concentration (CMC).

Biosurfactants include glycolipids (e.g. rhamnolipids (RLP), mannosylerythritol lipids (MEL), sophorolipids (SLP), cellobiose lipids and trehalose lipids), lipopeptides (e.g. surfactin, iturin, fengycin, and lichenysin), flavolipids (FL), fatty acids, phospholipids (e.g., cardiolipin, phosphatidylglycerol), and high molecular weight polymers such as lipoproteins, lipopolysaccharide-protein complexes, and polysaccharide-protein-fatty acid complexes.

Most biosurfactant-producing organisms produce biosurfactants in response to the presence of a hydrocarbon source (e.g. oils, sugar, glycerol, etc.) to facilitate uptake. Other media components, such as the concentration of iron, can affect biosurfactant production significantly. Microbial biosurfactants are produced by a variety of microorganisms such as bacteria, fungi, and yeasts. Non-limiting examples include Pseudomonas spp. (e.g., P. aeruginosa, P. putida, P. fluorescens, P. fragi, and P. syringae), Flavobacterium spp., Bacillus spp. (e.g., B. subtilis, B. pumilus, B. cereus, B. amyloliquefaciens and B. licheniformis), Campylobacter spp., Rhodococcus spp., Arthrobacter spp., Corynebacterium spp., Starmerella spp. (e.g., S. bombicola), Wickerhamomyces spp. (e.g., W. anomalus), Candida spp. (e.g., C. albicans, C. rugosa, C. tropicalis, C. lipolytica, C. glabrata, and C. torulopsis), Saccharomyces spp. (e.g., S. cerevisiae, S. chlororaphis), Pichia spp. (e.g., P. anomala and P. occidentalis) and Meyerozyma spp. (e.g., M. guilliermondii). The biosurfactant may be obtained by a fermentation process known in the art such as solid-state fermentation, submerged fermentation, or a combination thereof.

In specific embodiments, the adjuvant component comprises one or more glycolipid biosurfactants and/or one or more lipopeptide biosurfactants.

In certain preferred embodiments, the adjuvant component comprises a sophorolipid (SLP). SLP are glycolipid biosurfactants produced by, for example, various yeasts of the Starmerella clade. SLP consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). S. bombicola produces a specific enzyme, called S. bombicola lactone esterase, which catalyzes the esterification of linear SLP to produce lactonic SLP.

In one embodiment, the SLP is an acidic, or linear, SLP. The SLP could also be the lactonic form, a non-acetylated sophorolipid, a mono-acetylated sophorolipid, a di-acetylated sophorolipid, or any isoform thereof.

In certain embodiments, the biosurfactant is a RLP, such as, a mono-rhamnolipid, a di-rhamnolipid, or any other isoform thereof.

In certain embodiments, the biosurfactant is a MEL, such as MEL-A, MEL-B, MEL-C, or MEL-D, or any isoforms with varying fatty acid lengths and/or hydrophobic portions.

In certain embodiments, the biosurfactant is a trehalose lipid or any isoform thereof.

In certain embodiments, the biosurfactant is a lipopeptide. In certain preferred embodiments, the lipopeptide is surfactin, with a structure comprising a peptide loop of seven amino acids and a hydrophobic fatty acid chain that is thirteen to fifteen carbons long. Advantageously, the fatty acid chain allows for penetration of a cellular membrane. In specific embodiments, the amino acids comprise L-aspartic acid, L-glutamic acid, two L-leucines, two D-leucines, and L-valine.

In certain embodiments, the lipopeptide is an inturin. Iturins have amino acid residues that make up the peptide moiety and a variety of fatty acids or fatty acid derivatives that make up the hydrophobic tail of the biosurfactant. In preferred embodiments, the lipopeptide is iturin A with a structure comprising a peptide loop of seven amino acids, which are two D-asparagines, L-asparagine, D-tyrosine, L-glutamine, L-proline, and L-serine, and a β-amino fatty acid chain that can vary from fourteen to seventeen carbons long.

The MEL, trehalose lipid, rhamnolipid, sophorolipid, surfactin, or iturin, or any combinations thereof are preferably present in the subject adjuvant composition in therapeutically-effective amounts. In one embodiment, this means the biosurfactants are present in a critical micelle concentration (CMC). The CMC is the concentration of surfactants above which micelles will form and any additional surfactants that are added to the composition create additional micelles or are incorporated into existing micelles. Micelles or related variants of the micelle that can be used as a drug delivery system such as liposome, nanoparticles, or nanoemulsion droplets facilitate the methods of the subject invention.

In certain embodiments, a therapeutically-effective amount of biosurfactants in the composition is 0.001 to 90% to weight (wt %), preferably 50% or less, more preferably 25 wt % or less, even more preferably 10, 8, 5, 4, 3, or 2 wt % or less. In certain embodiments, the biosurfactant is present at more than 0.01, 0.02, 0.03, 0.05, 0.08, 0.1, 0.2, or 0.5%.

The one or more biosurfactants can further be chosen from: a modified form, derivative, fraction, isoform, or subtype of a biosurfactant, including forms that are naturally or artificially modified. The use of different isomers or forms of a biosurfactant can be beneficial in that the skilled artisan can tailor the adjuvant composition depending upon its interactions with a particular drug. That is, a certain isoform of a biosurfactant might be more effective with a certain drug due to, for example, the chemical structure of the compounds.

In some embodiments, the therapeutic composition comprises the adjuvant component pre-mixed with the active component. Alternatively, the adjuvant component can be separate from the active component, wherein the adjuvant component is intended to be administered concurrently with (e.g., 1, 5, 10, 15, 30, or 60 minutes or less before or after) the active component.

In one embodiment, the adjuvant component is formulated as a delivery system for a drug compound, wherein the biosurfactant(s) of the adjuvant component form a liposome, nanocapsule, microemulsion droplet, micelle or other biosurfactant-based delivery system with the drug compound encapsulated therein. In one embodiment, additional biological polymers can be included to provide further structure for the biosurfactant-based delivery system.

The biosurfactant-based delivery system can enhance the bioavailability, stability, and/or localization of a drug compound by a number of means. In certain embodiments, the delivery system protects the drug compound from components in the blood that might bind and prevent it from reaching a target site. In other embodiments, the delivery system inhibits the secretion of the compound by P-gp by affecting the membranes in which P-gp resides or preventing P-gp from recognizing the drug as a substrate. Additionally, in certain embodiments, the delivery system can prolong the half-life of drug compounds that might otherwise be degraded by acids or enzymes. This can facilitate oral administration of the drug compound, as it creates a barrier against acids or enzymes. Furthermore, in some embodiments, the delivery system formulation allows for timed release of the drug, thereby reducing the potential toxicity or side effects of the drug in a subject and/or decreasing the number of doses that must be administered.

In one embodiment, the subject therapeutic compositions are formulated as an orally-consumable product, such as, for example a food item, capsule, pill, or drinkable liquid. An orally deliverable drug is any drug delivered via initial absorption in the gastrointestinal tract or into the mucus membranes of the mouth. The subject compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable drug can be formulated into an orally consumable product, and an orally consumable product can comprise an orally deliverable drug, the two terms are not meant to be used interchangeably herein.

Orally consumable products include all substances or products intended to be ingested by humans or animals in a processed, semi-processed, or unprocessed state. This also includes substances that are added to orally consumable products (particularly food and drug products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

In certain embodiments, the subject therapeutic composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a drug composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In one embodiment, the therapeutic composition can be made into aerosol formulations so that, for example, it can be nebulized or inhaled. Suitable formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the therapeutic composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine. in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the therapeutic composition can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

In certain embodiments, the use of biosurfactants in the subject compositions decreases the amount of chemical surfactants (e.g., sodium dodecyl sulfate) needed for a drug compound to be effective. In some embodiments, the use of biosurfactants may eliminate the use of chemical surfactants altogether.

In certain embodiments, the use of biosurfactants in the subject compositions can increase the pre-administration stability of a drug compound, thereby decreasing the need for a robust cold chain to transport and store the composition before administration to a subject. The composition can be stored for an increased length of time at temperature between −20° C. and 4° C., at temperature at about 4° C., a temperature between 4° C. and room temperature, a temperature at about room temperature, or a temperature above room temperature but below 37° C.

In some embodiments, the use of biosurfactants in the subject compositions can increase the resistance of a drug to ultraviolet light degradation. For example, moxifloxacin, like other fluoroquinolones and tetracyclines, is sensitive to UV light exposure during storage. Upon exposure to UV light, the active compound can degrade, partially or entirely, depending on the intensity of the UV light, the time of exposure, and the accompany chemicals in the composition.

Further components can be added to the compositions as are determined by the skilled artisan such as, for example, buffers, carriers, viscosity modifiers, preservatives, flavorings, dyes and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

### Methods of Enhancing Efficacy of Health Compounds

The subject invention further provides a method of enhancing the bioavailability, stability, and/or localization of a drug compound in a subject in need thereof, wherein the drug compound is administered to the subject as part of a therapeutic composition according to the subject invention. In certain embodiments, the method can also be used for reducing the volume administered by unit of dosage of a drug compound that is required for it to be therapeutically-effective.

In some embodiments, bioavailability can be defined as the proportion of a drug administered that reaches systemic circulation unchanged. In preferred embodiments, bioavailability of a drug is enhanced by administering the drug with a therapeutically-effective amount of an adjuvant composition according to the subject invention.

The drug compound can be administered simultaneously with the adjuvant component, for example, as part of a single, pre-mixed composition. Alternatively, the drug compound can be administered separately from the adjuvant component. In this this alternative embodiment, the drug compound is administered either immediately before or immediately after the adjuvant composition is administered, wherein “immediately before” or “immediately after” means 60 minutes, 30 minutes, 15 minutes, 10 minutes, 5 minutes, 4 minutes, 3 minutes, 2 minutes, 1 minute, 30 seconds or less before or after the administration of the drug.

In preferred embodiments, the biosurfactants of the adjuvant composition are selected from, for example, glycolipids, lipopeptides, and any modified form, derivative, fraction, isoform, or subtype thereof. Combination of biosurfactants and their various forms are also envisioned.

As used herein, “administering” a composition refers to delivering it to a subject such that it contacts a target or site in which the composition can have an effect on that target or site. The effect can be due to, for example, the action of a drug compound, due to a biosurfactant composition, or because of a combined effect of the biosurfactant and drug compound. Administration can be acute or chronic (e.g., hourly, daily, weekly, monthly, etc.) or in combination with other agents. The subject compositions can be administered by any route of administration provided they are formulated for such a route. In this way, the therapeutic effects attainable by the methods and compositions of the invention can be, for example, systemic, local, tissue-specific, etc. depending on the specific needs of a given application of the invention.

In certain embodiments, the methods result in an increase in the solubility of a drug in an aqueous solution for administration to a subject. In exemplary embodiments, drugs that are soluble in concentrations less than 10 mg/ml are of particular interest. These drugs include, for example, azithromycin, moxifloxacin, bortezomib, lenalidomide, abiraterone acetate, erlotinib, aspirin, naproxen, ibuprofen, nitazoxanide, testosterone, sildenafil, vardenafil, tadalafil, indinavir, or any alternative form of these drugs. The alternative forms can comprise, for example, the various marketed forms of testosterone including unmodified and testosterone esters such as testosterone cypionate and testosterone propionate. By administering a therapeutically-effective dose of a drug and solubility-enhancing biosurfactant to a subject, a greater amount of the drug can be delivered per unit volume of dose. Additionally, due to increased solubility, the drug can move more easily into the circulatory system without being eliminated or crystallized.

In certain embodiments, the method can result in enhanced membrane permeability potential certain drugs, such as, for example, lenalidomide, abiraterone acetate, capecitabine, metformin, nitazoxanide, or any alternative form thereof through epithelial tissues in a subject. The administration of the subject therapeutic compositions facilitates the movement of the drugs, for example, from the GI tract to the circulatory system.

In certain embodiments, the method can result in increased movement of certain drugs, such as, for example, bortezomib, lenalidomide, donepezil, nitazoxanide, varenicline, sildenafil, vardenafil, tadalafil, indinavir, or any alternative form thereof from the circulatory system through the BBB or blood-testis barrier. Sildenafil has been demonstrated as a phosphodiesterase 5 inhibitor in the brain, potentially limiting the effects of Alzheimer's disease with treatment using this pharmaceutical. The testes and brain can be reservoirs for viruses, including retroviruses. With enhanced penetration through these barriers, indinavir can eliminate the viruses and sildenafil can enhance brain function.

In certain embodiments, the method can result in increased elimination half-life of certain drugs, such as, for example, clindamycin, capecitabine, metformin, nitazoxanide, indinavir, or any alternative thereof in the subject. These drugs have demonstrated elimination half-lives in humans from 33 minutes to 8.7 hours, necessitating that they be administered 1 to 4 times each day. The micelles created by the biosurfactants in the administered compositions can have a number of mechanisms that increase the elimination half-life including, but not limited to, increasing solubility of the drugs, increasing the permeability of the drugs, and prolonging the delivery of drugs by delivering them in a micelle or other related biosurfactant aggregate. These and other mechanisms may be employed in combination or individually to increase the elimination half-life of the drugs.

In certain embodiments, the method can result in increased time between the administration of doses of certain drugs, such as, for example, daptomycin, clindamycin, azithromycin, moxifloxacin, lenalidomide, abiraterone acetate, capecitabine, erlotinib, aspirin, naproxen, ibuprofen, metformin, donepezil, nitazoxanide, varenicline, sildenafil, vardenafil, tadalafil, indinavir, or any alternative form thereof. Each of these drugs is, or can be, prescribed to be taken more than once daily. In accordance with the subject invention, the method of administering these drugs to a subject can increase the time between doses by enclosing the drugs in biosurfactant aggregates. After administration to a subject, the biosurfactants gradually decrease in concentration in the subject. As the relative biosurfactant concentration decreases to the CMC and eventually below the CMC, the drug is released steadily. This discharges the drug in a manner more slowly than the conventional “all-at-once” administration, facilitating a less frequent dose administration of each drug.

In certain embodiments, the method can result in inhibition of secretion systems in the subject when administering the adjuvant component with drugs such as, for example, daptomycin, clindamycin, moxifloxacin, bortezomib, lenalidomide, capecitabine, doxorubicin, erlotinib, sildenafil, vardenafil, tadalafil, indinavir, or any alternative form thereof. P-gp is a secretion system that can be suppressed by biosurfactant compositions according to the subject invention. The biosurfactant(s) can alter the subject's membrane lipids, affecting the functioning of P-gp that relies on an intact, stable cell membrane. In some embodiments, the effects of the administration of biosurfactants with the drug are not limited to P-gp inhibition but may also entail reversible inhibition.

In certain embodiments, the method can result in decreased latency period to achieve the maximum concentration of certain drugs, such as, for example, donepezil, or any alternative form thereof, in the cerebral spinal fluid. The biosurfactant(s) can facilitate an increased rate of accumulation and greater persisting concentration of the drug in the cerebral spinal fluid of a subject. Previous maximum cerebral spinal fluid concentrations in subject could take up to 2 years to achieve with ordinary drug administration.

Each drug exemplified in the present invention has intended uses; however, new research can often change or add to the initial desired treatment purpose. Because of research into the repurposing of FDA approved drugs, the examples of treatments exemplified in this disclosure are non-limiting. Daptomycin, clindamycin, azithromycin, and moxifloxacin are all antibiotic compounds. Clindamycin and Azithromycin inhibit protein synthesis by binding 50S rRNA. Daptomycin disrupts the negatively charged phospholipids in bacterial cell membranes. Moxifloxacin inhibits DNA gyrase and topoisomerase. Azithromycin is currently being investigated for use as an anti-malarial. Bortezomib, lenalidomide, abiraterone acetate, capecitabine, doxorubicin, and erlotinib are all anti-cancer pharmaceuticals. Pegfilgrastim is often taken with anti-cancer treatments to stimulate the bone marrow to produce more neutrophils. Additionally, bortezomib is useful in treating systemic lupus erythematosus, and erlotinib has been used as an antiviral. Aspirin, naproxen, and ibuprofen are nonsteroidal anti-inflammatory drugs (NSAIDs) that are used to treat pain and pyrexia through the inhibition of cyclooxygenase. Aspirin is often taken to prevent cardiovascular disease. Both aspirin and ibuprofen have demonstrated indirect antimicrobial properties. Metformin is used to treat type 2 diabetes and polycystic ovary syndrome, and more recently, it has been shown to be useful in treating numerous dermatological conditions including acne and psoriasis. Donepezil is used to treat Alzheimer's disease and more recently has been shown useful in treating multiple sclerosis. Nitazoxanide is an anti-parasitic drug that may also suppress glioblastoma. Varenicline is an agonist of the nicotinic acetylcholine receptor, and may improve cognition associated with aging and schizophrenia. Testosterone is used to treat male hypogonadism and certain types of breast cancers. Sildenafil, vardenafil, and tadalafil are all treatments of erectile dysfunction and pulmonary arterial hypertension. Tadalafil has been shown to also treat benign prostatic hyperplasia, reverse tumor specific immune suppression, and inhibit P-gp in P-gp over synthesizing cancer cells. Sildenafil also inhibits P-gp secretion in cancer cells and has been used to treat breast cancer with doxorubicin, inhibit colorectal cancer cells, and is a phosphodiesterase inhibitor in the brain. Indinavir is an antiretroviral drug that may also be useful in treating Ebola viral infection.

1. A composition comprising an active component and an adjuvant component, the active component comprising a drug compound, a vitamin, a mineral, a supplement, an enzyme and/or an herbal extract;
wherein said adjuvant component comprises an efficacy-enhancing amount of a biosurfactant; and
wherein stability of the active component is enhanced compared to a composition comprising the same active component without the adjuvant component.

2. (canceled)

3. The composition of claim 1, wherein the biosurfactant is selected from lipopeptides and glycolipids.

4. The composition of claim 3, wherein the glycolipids are selected from sophorolipids, rhamnolipids, trehalose lipids, cellobiose lipids and mannosylerythritol lipids.

5. (canceled)

6. The composition of claim 1, wherein a biosurfactant is present in critical micelle concentration (CMC).

7. The composition of claim 1, formulated for administration to a subject through a route selected from oral, injection, rectal, vaginal, ocular, aural, nasal, mucosal, inhalation, nebulization, and dermal.

8. The composition of claim 1, formulated as a biosurfactant-based delivery system, wherein the drug is encapsulated in a biosurfactant-based particle.

9. The composition of claim 1, wherein the active component comprises a pharmaceutical or OTC drug compound selected from daptomycin, clindamycin, azithromycin, moxifloxacin, bortezomib, lenalidomide, abiraterone acetate, pegfilgrastim, capecitabine, doxorubicin, erlotinib, aspirin, naproxen, ibuprofen, metformin, donepezil, nitazoxanide, varenicline, testosterone, sildenafil, vardenafil, tadalafil, and indinavir.

10. The composition of claim 1, wherein the active component comprises a vitamin selected from vitamin A, D, E and K.

11. The composition of claim 1, wherein the active component comprises cannabidiol.

12. The composition of claim 1, wherein the active component comprises an enzyme selected from CoQ10 and nattokinase.

13. A method for enhancing the stability, of an active component selected from a drug compound, a vitamin, a mineral, a supplement, an enzyme and an herbal extract, the method comprising administering to a subject a therapeutically-effective amount of said active component simultaneously or sequentially with an adjuvant component comprising a biosurfactant.

14. The method of claim 13, wherein the solubility of the active component is increased.

15. (canceled)

16. The method of claim 13, wherein the elimination half-life of the active component is enhanced.

17. (canceled)

18. A method for decreasing the latency period to achieve a maximum therapeutic concentration of an active component in a subject comprising administering to a subject a therapeutically-effective amount of said active component simultaneously or sequentially with an adjuvant component comprising a biosurfactant.

19-21. (canceled)

22. The method of claim 13, wherein the active component is a pharmaceutical or OTC drug compound selected from one or more of daptomycin, clindamycin, azithromycin, moxifloxacin, bortezomib, lenalidomide, abiraterone acetate, pegfilgrastim, capecitabine, doxorubicin, erlotinib, aspirin, naproxen, ibuprofen, metformin, donepezil, nitazoxanide, varenicline, testosterone, sildenafil, vardenafil, tadalafil, and indinavir.

23. The method of claim 22, wherein the solubility of the azithromycin, moxifloxacin, bortezomib, lenalidomide, abiraterone acetate, erlotinib, aspirin, naproxen, ibuprofen, nitazoxanide, testosterone, sildenafil, vardenafil, tadalafil, and/or indinavir is increased in a formulation to be administered to the subject.

24. The method of claim 22, wherein the solubility of the azithromycin, moxifloxacin, bortezomib, lenalidomide, abiraterone acetate, erlotinib, aspirin, naproxen, ibuprofen, nitazoxanide, testosterone, sildenafil, vardenafil, tadalafil, and/or indinavir is increased in the subject.

25. (canceled)

26. The method of claim 22, wherein the elimination half-life of clindamycin, capecitabine, metformin, nitazoxanide, and/or indinavir, is enhanced.

27-29. (canceled)

30. A method for decreasing the latency period to achieve the maximum therapeutic concentration of donepezil or any alternative form of donepezil in the cerebral spinal fluid in a subject comprising administering to a subject a therapeutically-effective amount of said donepezil or any alternative form of donepezil simultaneously or sequentially with an adjuvant component comprising a biosurfactant.

31. The method of claim 13, wherein the active component is a vitamin selected from vitamin A, D, E and K.

32. The method of claim 13, wherein the active component is cannabidiol.

33. The method of claim 13, wherein the active component is an enzyme selected from CoQ10 and nattokinase.

34. The method of claim 13, wherein use of a biosurfactant as the adjuvant component decreases or eliminates the need for use of chemical surfactants in drug and supplement formulations.

35. The method of claim 13, further comprising storing the active component mixed with the adjuvant component for a period of time, wherein the stability of the active component is enhanced such that the active component remains therapeutically-effective throughout storage.

36. The method of claim 35, wherein stability is enhanced when storage occurs at temperatures between −20° C. and 37° C.

37. The method of claim 35, wherein stability is enhanced by enhancing the active component's resistance to ultraviolet light degradation.

38. A composition comprising a drug compound, a vitamin, a mineral, a supplement, an enzyme or an herbal extract, and an efficacy-enhancing amount of a biosurfactant adjuvant, wherein the drug compound, vitamin, mineral, supplement, enzyme or herbal extract has enhanced stability compared to a composition comprising the same drug compound, vitamin, mineral, supplement, enzyme or herbal extract without the biosurfactant adjuvant.

39. The method of claim 35, wherein the adjuvant increases a shelf life of said composition.
